(19) United States
(12) Patent Application Publication
(10) Pub. No.: US 2018/0206842 A1
(43) Pub. Date: Jul. 26, 2018

(54) SUTURE CARRIER DEVICES, SYSTEMS AND METHODS OF USING SAME

(71) Applicant: Surgical Specialities Corporation, Braintree, MA (US)

(72) Inventors: Angela Wentling, Perkiomenville, PA (US); David Mucko, Lancaster, PA (US); Juan Arango, Gainesville, FL (US)

(21) Appl. No.: 15/322,419

(22) PCT Filed: Jul. 21, 2015
(86) PCT No.: PCT/US2015/041393

§ 371 Date: Dec. 27, 2016

Related U.S. Application Data
(60) Provisional application No. 62/027,022, filed on Jul. 21, 2014.

(51) Int. Cl. A61B 17/06

(52) U.S. Cl. CPC A61B 17/06133; A61B 2017/00526

(57) ABSTRACT

The present disclosure is directed towards an apparatus for holding a suture. The apparatus may comprise a carrier including a base. The carrier may be configured to retain a cover over the base. The base may be configured to mount the carrier on a fixture and include a primary wrapping path disposed thereon, the wrapping path for wrapping at least a portion of a suture thread. The apparatus may include a plurality of suture retaining canopies located at an outer portion of the primary wrapping path and projecting from the base. The suture retaining canopies may be configured to facilitate retaining a suture thread within the primary suture path. The apparatus may also include a needle retainer disposed on the base. The needle retainer may include at least one slot configured to retain a needle.

### CROSS-REFERENCE TO RELATED APPLICATION

This application claims the benefit under 35 U.S.C. § 119(e) of U.S. Provisional Patent Application No. 62/027,022, filed Jul. 21, 2014, which application is hereby incorporated by reference in its entirety.

### BACKGROUND

### Technical Field

This disclosure relates to suture packaging devices and methods for packing sutures. More particularly, this disclosure relates to tray carriers and methods for packaging sutures in tray carriers.

### Description of the Related Art

Wound closure devices such as sutures, staples, and tacks have been widely used in surgical procedures in humans and animals for closing wounds, repairing traumatic injuries or defects, joining tissues together, attaching foreign elements to tissues, repositioning tissues to new anatomical locations, and a myriad of other purposes.

Sutures are also often used as wound closure devices. Sutures typically consist of a filamentous suture thread attached to one or more needles having a sharp point. Suture threads can be made from a wide variety of materials including bioabsorbable (i.e., that break down completely in the body over time), or non-absorbable (permanent; non-degradable) materials. Absorbable sutures have been found to be particularly useful in situations where suture removal might jeopardize the repair, or where the natural healing process renders the support provided by the suture material unnecessary after wound healing has been completed; as in, for example, completing an uncomplicated skin closure. Non-degradable (non-absorbable) sutures are used in wounds where healing may be expected to be protracted or where the suture material is needed to provide physical support for long periods of time; as in, for example, deep tissue repairs, high tension wounds, many orthopedic repairs and some types of surgical anastomosis.

Like any long flexible filament, sutures may tangle during removal from their packaging, when preparing them for use, or during use. Because of the tendency for long flexible filaments to tangle, manufacturers and medical practitioners use great caution to prevent tangling during the packaging process and during use of sutures. The handling of sutures and their attached needles also complicates the packaging and use of sutures.

Improperly packaged sutures may place the needles in a position where it is more likely to cause harm to medical practitioners, and some packaging device designs and methods for packaging may place the needles in less secure or less safe positions.

Suture packages, and in particular plastic tray suture carriers have only one path in which to wrap a suture. The single path typically restricts suture wrapping into circular or elliptical patterns and can inhibit the use of sutures other than simple geometry sutures with a single needle attached to a single straight thread.

Sutures, particularly sutures with synthetic thread may have a straight set in which the thread, when bent or wrapped, tends to unwrap and spring back towards a straight state. The thread set may cause sutures to prematurely unwind or tangle during wrapping on, transport in, and removal from a suture package.

### BRIEF SUMMARY

Embodiments described herein provide a packaging device that may safely and securely hold a suture and its needles and provides for its easy and safe removal and a suture carrier that may retain the suture thread and resist its tendency to unwrap. In addition the embodiments describe safe, easy, and efficient methods for loading and removing sutures from their packaging.

The present disclosure is directed towards an apparatus for holding a suture. The apparatus may comprise a carrier including a base. The carrier may be configured to retain a cover over the base. The base may be configured to mount the carrier on a fixture and include a primary wrapping path disposed thereon, the wrapping path for wrapping at least a portion of a suture thread. The apparatus may include a plurality of suture retaining canopies located at an outer portion of the primary wrapping path and projecting from the base. The suture retaining canopies may be configured to facilitate retaining a suture thread within the primary suture path. The apparatus may also include a needle retainer disposed on the base. The needle retainer may include at least one slot configured to retain a needle.

The present disclosure is also directed to a method of using a suture carrier. The method may include loading a suture carrier onto a fixture by aligning one or more fixture apertures with a corresponding one or more fixture pins and lowering the suture carrier onto the wrapping fixture, wrapping a first suture thread into a first loop and placing the first suture thread on a base of the suture carrier, positioning at least a portion of the first loop of the first suture thread under a suture canopy, installing a cover over the first suture and the suture carrier, and engaging the cover with at least a portion of the suture carrier.

### BRIEF DESCRIPTION OF THE SEVERAL VIEWS OF THE DRAWINGS

FIG. 1 shows an isometric view of an embodiment of a suture carrier;

FIG. 2 shows a top view of an embodiment of a cover for a suture carrier;

FIG. 3 shows an isometric view of an embodiment of a suture wrapping and packaging fixture;

FIG. 4 shows a bi-lateral cross-sectional view of a suture wrapping and packaging fixture;

FIG. 5 shows portions of a method for wrapping and packing a suture in a suture carrier according to one or more embodiments;

FIG. 6 shows portions of a method for wrapping and packing a suture in a suture carrier according to one or more embodiments;

FIG. 7 shows portions of a method for wrapping and packing a suture in a suture carrier according to one or more embodiments;

FIG. 8 shows portions of a method for wrapping and packing a suture in a suture carrier according to one or more embodiments;

FIG. 8-1 shows portions of a method for wrapping and packing a suture in a suture carrier according to one or more embodiments;

FIG. 9 shows portions of a method for wrapping and packing a suture in a suture carrier according to one or more embodiments;

FIG. 10 shows portions of a method for wrapping and packing a suture in a suture carrier according to one or more embodiments;

FIG. 11 shows portions of a method for opening and removing a suture from a suture carrier according to one or more embodiments;

FIG. 12 shows portions of a method for opening and removing a suture from a suture carrier according to one or more embodiments

FIG. 13 shows an isometric view of an embodiment of a suture carrier according to one or more embodiments.

### DETAILED DESCRIPTION

In the following description, certain specific details are set forth in order to provide a thorough understanding of various embodiments of the invention. However, one skilled in the art will understand that the invention may be practiced without these details. In other instances, well-known structures and steps associated with suture carriers and packaging have not been shown or described in detail to avoid unnecessarily obscuring descriptions of the embodiment of the invention.

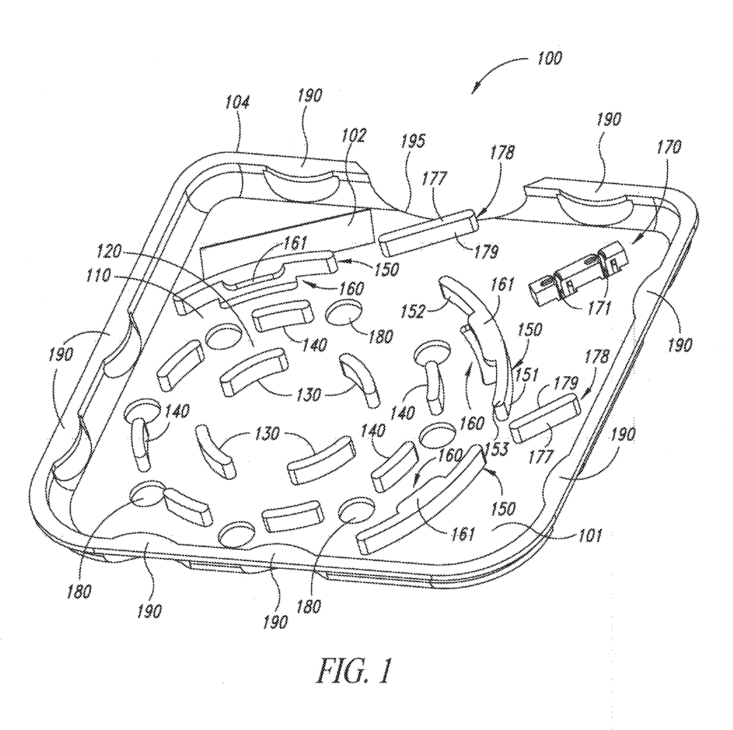

FIG. 1 shows an embodiment of a suture carrier for packaging and transporting one or more sutures. Suture carriers may come in a variety shapes and sizes. The suture carrier 100 shown in FIG. 1 is an embodiment of a plastic tray carrier. A plastic tray carrier may be made from a medical grade plastic compatible with ethylene oxide or gamma sterilization. A suture carrier 100 may include a number of features, for example, the suture carrier 100 may include one or more suture thread wrapping paths 110, 120 separated by carrier walls 130, 140, 150, and a suture needle retainer 170.

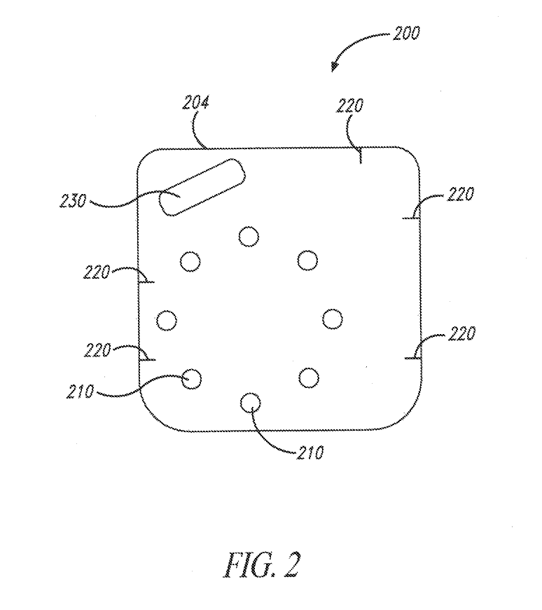

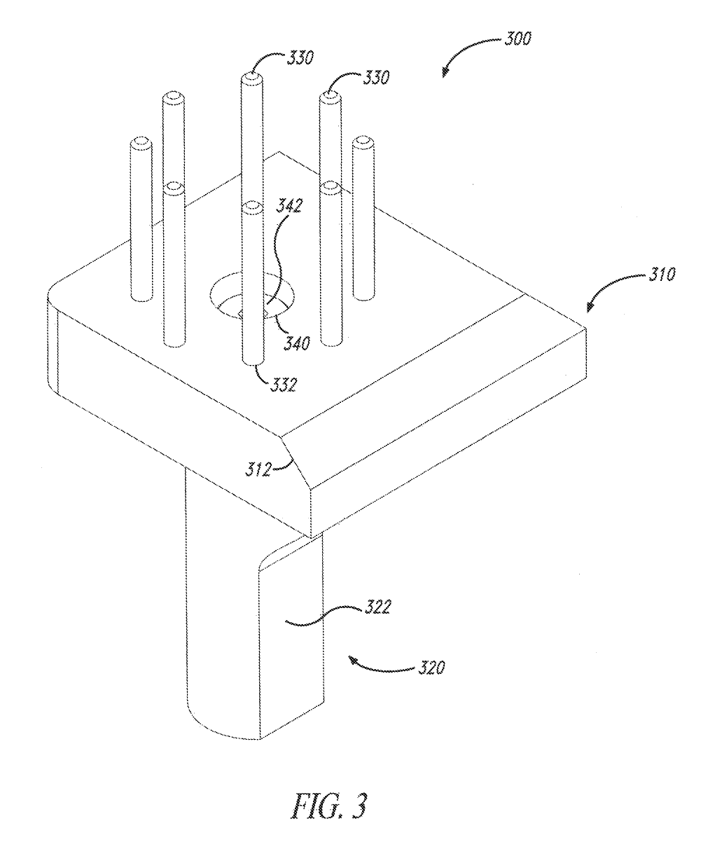

The suture carrier 100 may also include carrier holes 180 to facilitate retaining the suture carrier 100 on a wrapping fixture, for example a wrapping fixture as shown in FIG. 3, and cover tabs 190 for retaining a suture carrier cover, for example a suture carrier cover 200 as shown in FIG. 2.

One use of a suture carrier 100 may be to securely and safely store a suture needle and thread and provide a medical practitioner with easy and safe removal of a suture. The needle retainer 170 is adapted to hold one or more suture needles. For example, the illustrated needle retainer 170 includes two slots 171. Each slot 171 may hold one or more needles.

In some embodiments, a slot 171 may retain multiple needles stacked one on top of another or side-by-side within a single slot. Although depicted as having two slots 171, the needle retainer 170 may include a single slot or more than two slots. In some embodiments, the needle retainer may not have any slots and may retain suture needles using other structures. In some embodiments, a needle retainer may be adapted to hold large or small needles in correspondingly wide or narrow slots.

Although the illustrated suture carrier 100 includes a single needle retainer 170, a suture carrier may include more than one needle retainer. In some embodiments, a needle retainer may not be necessary, in which case, the suture carrier may not have a needle retainer. Moreover, although the needle retainer 170 is shown in the upper right-hand corner of suture carrier 100 and outside the outer perimeter of the wrapping paths 110, 120, as shown in FIG. 1, the needle retainer 170 may be located in any location that facilitates the retention of suture needles and the safe storage and use of sutures with a suture carrier.

A suture carrier may include needle walls, for example, the suture carrier 100 includes two needle walls 178. The needle walls 178 can help separate the suture needles from the suture thread and thereby reduce the risk of sharp needles damaging the suture thread and, therefore, they may be located near the needle retainer 170. For example, the illustrated suture carrier 100 includes two needle walls 178 on opposing sides of the needle retainer 170.

The needle walls 178 may be integrated into the suture carrier 100. As shown in FIG. 1, the needle walls 178 can protrude or extend upward from a base 101 of the suture carrier 100. In some embodiments, a mold or die forms the needle walls 178 from the suture carrier blank during the manufacturing process. For example, a mold set may include one mold with protrusions that correspond to cavities within a second matching mold. When the molds are pushed together with a blank between them, the protrusions force the carrier blank material into the cavities and thereby create the needle walls 178.

In some embodiments, the needle walls 178 are separate from the base 101 and may be adhered or otherwise coupled to the base 101 during the manufacturing process.

Although the needle walls 178 are shown as substantially straight formations on the carrier base, the needle walls 178 may be curved or may have multiple sections, wherein one or more sections are straight and one or more sections are curved. The opposing surfaces 179, 177 of the needle walls 178 may be substantially planar and parallel to one another and they may extend substantially perpendicular to the base 101. In some embodiments, the surfaces 179, 177 may not be parallel to each other. For example, a bottom portion of the surfaces 179, 177 at or near the base 101 may be further away from each other as compared to a top portion of the surfaces 179, 177 at a distance from the base 101.

The suture carrier 100 may also include the carrier walls 130, 140, 150. The carrier walls 130, 140, 150 may define wrapping paths in the suture carrier 100. For example, the outer carrier walls 150 and intermediate carrier walls 140 can define a primary wrapping path 110 while the intermediate carrier walls 140 and inner carrier walls 130 can define a secondary wrapping path 120. The outer carrier walls 150 may define an outer boundary of the primary wrapping path 110 while the intermediate carrier walls 140 may define an inner boundary of the primary wrapping path 110.

The carrier walls 130, 140, 150 may include multiple surfaces 151, 152, 153. The surfaces 151, 152, 153 may include, for example, an outward facing surface 151, an inward facing surface 152 and a top surface 153. The surfaces 151, 152 of the carrier walls may be substantially planar and parallel to one another and they may extend substantially perpendicular to the base 101. In some embodiments, the surfaces 151, 152 may not be parallel to each other. For example, a bottom portion of the surfaces 151, 152 at or near the base 101 may be further away from each other as compared to a top portion of the surfaces 151, 152 at a distance from the base 101.

The embodiment shown in FIG. 1 includes carrier walls 130, 140, 150 that protrude substantially perpendicularly upward from the base 101. In some embodiments, the carrier walls 130, 140, 150 may protrude at an angle other than a right angle from the base 101. For example, in some embodiments, the carrier walls 130, 140, 150 may be formed by slitting or cutting through the base 101 and folding a portion of the base 101 in an upward direction. The needle walls may include similar structure and be similarly formed.

The suture carrier 100 may also include one or more suture retention canopies 160. A suture retention canopy may be adapted or configured to retain a suture thread, in particular to retain a suture thread within a corresponding suture path. As shown in FIG. 1, the outer carrier walls 150 may include suture retention canopies 160 to retain suture thread within the primary suture wrapping path 110.

The suture retention canopies 160 may be adapted to include a ceiling or retaining wall 161 that extend towards or into the suture wrapping path associated with the carrier wall the canopy is attached to, for example, the retaining wall 161. Thus, the suture retention canopies 160 may extend from the outer carrier wall 150 into or over the primary suture wrapping path 110.

As shown in FIG. 1, the retaining wall 161 may extend from a carrier wall 150 in a direction parallel to the base 101. This arrangement may aid in retaining suture thread, particularly sutures with synthetic thread which may have a straight set. When bent or wrapped, a thread with a straight set tends to unwrap and spring back towards a straight state. The thread set and the tendency to unwrap or spring back towards a straight state may cause the thread to push outward or upward against the surface 152 of the carrier wall 150, the underside of the suture canopy 160, and its retaining wall 161. In this way, the base 101, carrier wall 150, and suture retention canopy 160 may facilitate the retention of a suture thread within the carrier 100 and, more particularly, within the primary wrapping path 110.

The suture carrier 100 may also include carrier holes 180. In some embodiments, for example as shown in FIG. 1, the carrier holes 180 may be located proximate the intermediate carrier walls 140 or otherwise proximate an inner boundary of a wrapping path.

Although depicted on the outer carrier walls 150 along the primary wrapping path 110, the suture retention canopies 160 may facilitate retaining suture thread within other thread wrapping paths, for example the secondary wrapping path 120, and may extend from other carrier walls 130, 140.

Although shown as part of the carrier walls 150, the suture retention canopies 160 may be independent of carrier walls. For example, a suture carrier 100 may include suture retention canopies 160 at locations between the carrier walls, for example, between one or more outer carrier walls 150 or between opposing carrier walls, for example, between the outer carrier walls 150 and the intermediate carrier walls 140.

In some embodiments, the suture retention canopy 160 may extend directly from the base 101 or other structure of the suture carrier 100. In such embodiments, a retaining canopy may be formed by slitting or cutting through the base 101 and folding a portion of the base 101 in an upward direction, out of the page as shown in FIG. 1.

Although the suture canopy retaining walls 161 are shown as parallel to the base 101, in some embodiments, a canopy may not be parallel to a base. For example, a retaining canopy or canopy retaining wall formed by slitting or cutting through a base and then folding a portion of the base in an upward direction may extend from the base at an angle less than 90 degrees or less than perpendicular to the base.

The carrier walls 130, 140, 150 may be integrated into the suture carrier 100. As shown in FIG. 1, the carrier walls 130, 140, 150 protrude or extend upward from the base 101 of the suture carrier 100. In some embodiments, a mold or die forms the carrier walls 130, 140, 150 from the suture carrier blank during the manufacturing process. For example, a mold set may include one mold with protrusions that correspond to cavities within a second matching mold. When the molds are pushed together with a blank between them, the protrusions force the carrier blank material into the cavities and thereby create the carrier walls 130, 140, 150. In some embodiments, the suture carrier may be injection molded or formed using any technique known in the art.

In some embodiments, the carrier walls 130, 140, 150 are separate from the base 101 and may be adhered or otherwise coupled to the base 101 during the manufacturing process.

Although the carrier walls 130, 140, 150 are shown as substantially straight formations on the carrier base, the carrier walls 130, 140, 150 may be curved or may have multiple sections, wherein one or more sections are straight and one or more sections are curved. The surfaces 151, 152 of the carrier walls 130, 140, 150 may be substantially planar and parallel to one another and they may extend substantially perpendicular to the base 101. In some embodiments, the surfaces 151, 152 may not be parallel to each other. For example, a bottom portion of the surfaces 151, 152 at or near the base 101 may be further away from each other as compared to a top portion of the surfaces 151, 152 at a distance from the base 101.

In some embodiments, the carrier walls 130, 140, 150 may include a single surface 151, 152. In such embodiments, a surface may be formed by slitting or cutting through the base 101 and folding a portion of the base 101 in an upward direction, out of the page as shown in FIG. 1.

The suture carrier 100 may include a base 101, which may include one or more carrier holes 180. The carrier holes 180 may be apertures cut, punched, or otherwise formed through the base 101. The carrier holes 180 allow one or more fixture pins, such as fixture pins 330, shown in FIG. 3, to pass through the carrier holes 180.

Although the embodiment shown in FIG. 1 depicts a suture carrier 100 with eight round carrier holes 180, in some embodiments there may be more or fewer carrier holes 180 or the carrier holes 180 may have other shapes or configurations. For example, in some embodiments four, six, or nine holes may be used and in some embodiments, the fixture holes may have a square or octagon all shape or any shape that is compatible with a corresponding fixture.

Fixture pins may facilitate wrapping a suture thread and packaging it in the suture carrier 100. For example, up to eight fixture pins may be inserted through the eight carrier holes 180. An operator may then wrap a portion of a suture thread around the fixture pins. An operator could then place the portion of the suture thread wrapping around the fixture pins into the primary wrapping path.

In some embodiments, fixture pins are not used as a guide for wrapping suture thread. For example, an operator may wrap a portion of suture thread within the secondary wrapping path 120 without the use of fixture pins.

In some embodiments, a first portion of one or more suture threads may be wrapped and placed within the primary wrapping path 110 while a second portion of the one or more suture threads may be wrapped and placed within the secondary wrapping path 120. In some embodiments, a first suture thread may be wrapped in the primary wrapping path 110 while a second suture thread may be wrapped in the secondary wrapping path 120.

The suture carrier 100 may include a perimeter wall 104 around the perimeter of the suture carrier 100. The perimeter wall 104 may project upwards and out from the base of the suture carrier 100. The perimeter wall 104, may extend completely or partially around the perimeter of the suture carrier 100. For example, the illustrated suture carrier 100 includes a cutout 195 that interrupts the perimeter wall 104. In some embodiments, the suture carrier 100 may include a cutout 195 that does not interrupt the perimeter wall 104. For example, the perimeter wall may continue along an edge of the cutout 195.

The suture carrier 100 may also include cover tabs 190. The cover tabs 190 may extend out from the perimeter wall 104 such that they overhang the interior of the suture carrier 100. The cover tabs 190 are configured such that a perimeter of a suture cover, for example, a perimeter 204 of the suture cover 200 in FIG. 2, may engage with an underside or engagement surface of the cover tabs 190.

Although the cover tabs 190 of suture carrier 100 extend from the perimeter wall 104, in some embodiments, cover tabs may be independent of a perimeter wall. For example, the cover tabs may extend directly from the perimeter of a suture carrier.

The perimeter wall 104 adds depth to the suture carrier 100 and may have a height greater than the height of the carrier walls 130, 140, 150, and the needle retainer 170. The height of the perimeter wall 104 may be selected such that it is greater than the combined height of the carrier walls 130, 140, 150, the thickness of the suture carrier cover 200, and the thickness of the cover tabs.

In some embodiments, the distance between the base of the suture carrier 100 and the underside of the cover tabs 190 is about the same height as the carrier walls 130, 140, 150 or the needle retainer 170. In some embodiments, the distance between the base of the suture carrier 100 and the underside of the cover tabs 190 is about the same height as the combined height of the carrier walls 130, 140, 150 or the needle retainer 170 and the thickness of the suture carrier cover 200.

In some embodiments, the location or height of the cover tabs may be such that when a suture carrier cover 200 engages with an underside of the cover tabs 190, the suture carrier cover 200 contacts the top of one or more carrier walls 130, 140, 150 or needle retainers 170. This may cause bending in the suture carrier cover 200, which may reduce the suture carrier cover's movement after installation, and may facilitate a more positive hold on the suture carrier cover. A more positive hold may help prevent the carrier cover 200 from disengaging with the cover tabs 190.

In some embodiments, the location or height of the cover tabs may be such that an installed suture carrier cover 200 is only loosely engaged between the underside of the cover tabs 190 and the tops of the carrier walls 130, 140, 150, or the needle retainer 170.

Returning now to the suture carrier cutout 195, the suture carrier cutout 195 may provide access to the underside of an installed suture carrier cover 200. For example, in removing a suture carrier cover 200, the suture carrier cutout 195 may provide space for a practitioner to place their fingers such that the practitioner may grasp the suture carrier cover 200 from the underneath as well as from above. In this way, the practitioner may grab suture carrier cover 200, disengage it from the cover tabs 190, and remove the suture carrier cover 200 from the suture carrier 100. In some embodiments, the suture carrier 100 may include more than one suture carrier cutout 195.

As shown in FIG. 1, a needle wall 178 may protect an operator from pushing their finger into the suture carrier 100 and inadvertently contacting the point of a needle. In some embodiments, a needle wall 178, or other structure, such as a perimeter wall 104, may extend along some or all of the edge of the suture carrier cutouts 195. In some embodiments, rather than extending along an edge of the suture carrier cutouts 195, the structure may be located in proximity to the suture carrier cutouts 195 and between the suture carrier cutouts 195 and a potential hazard, such as the point of a needle, within the suture carrier 100.

The suture carrier 100 may also include a location for product information, for example, a recess 102 which is sized and shaped to receive a product information label.

FIG. 2 shows an embodiment of a suture carrier cover 200. The suture carrier cover 200 can cover and protect the contents of the suture carrier 100 by acting as a barrier against external contaminants and other objects. The cover 200 can also protect users from the contents of the suture carrier 100 by covering the needles or other sharp objects within the suture carrier 100.

The suture carrier cover 200 may include wrapping fixture apertures, such as retention and alignment cover apertures 210. The cover apertures 210 may be cut, punched, or otherwise formed through the cover 200. The cover apertures 210 allow one or more fixture pins, such as fixture pins 330, shown in FIG. 3, to pass through the cover 200.

Although the embodiment shown in FIG. 2 depicts a carrier cover 200 with eight round cover apertures 210, in some embodiments there may be more or fewer cover apertures 210 or the cover apertures 210 may have other shapes or configurations. For example, in some embodiments, four, six, or nine holes may be used and, in some embodiments, the fixture holes may have a square or octagon all shape or any shape that is compatible with a corresponding fixture.

The cover 200 may also include a perimeter 204. The perimeter 204 may be sized and shaped to correspond with a perimeter of a suture carrier, for example, the perimeter 104 of the suture carrier 100 shown in FIG. 1. In some embodiments, the perimeter 204 may be smaller than the perimeter of a corresponding suture carrier such that the cover 200 may fit within the boundary defined by a suture carrier perimeter.

The cover 200 may also include through slits 220. The through slits 220 may be positioned along or around the perimeter 204 of the cover 200. The through slits 220 may be cuts through the cover 200. The cut may start at an edge or perimeter 204 of the cover 200 and may extend towards the interior of the cover 200. In some embodiments, the through slits 220 may be perpendicular to an edge or perimeter of the cover 200.

The through slits 220 may create weak points in the cover 200. The location of the through slits 220 may correspond with one or more locations of cover tabs 190 of a suture carrier 100. By weakening the cover in locations that correspond to cover tabs, the through slits may facilitate easier installation of the cover 200 onto a suture carrier 100.

The suture carrier cover 200 may also include a cutout 230, to provide access to the recess 102 of the suture carrier 100. For example, the cutout 230 may permit a user to view a label or other information printed in the recess 102.

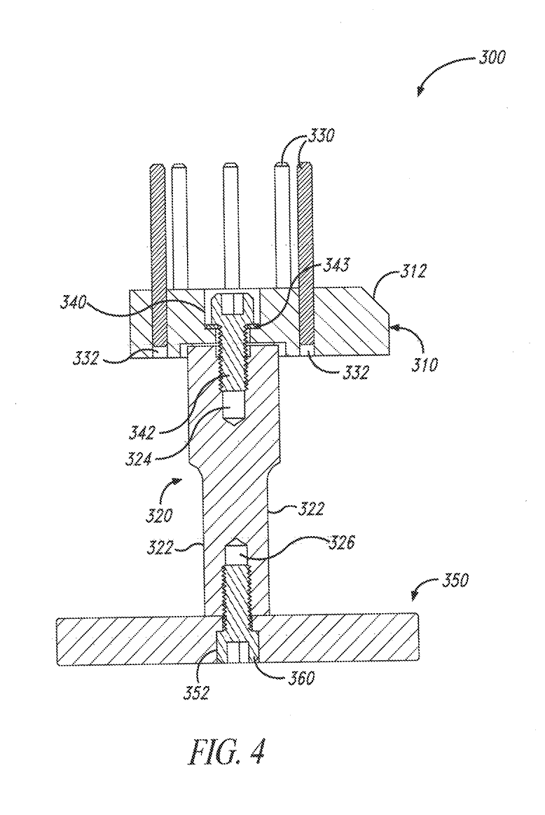

FIGS. 3 and 4 depict an embodiment of a wrapping fixture. Wrapping fixture 300 may be comprised of a mounting post 320, a mounting plate 310, pins 330, a base 350, a first coupler 342, and a second coupler 360.

The mounting post 320 includes one or more indexes or flats 322 for coupling a wrapping fixture 300 to a fixture holder, not shown, or to the base 350. The flats may interface with the fixture holder to aid in preventing the wrapping fixture 300 from rotating while coupled to or held by the fixture holder.

The first coupler 342 may couple the mounting plate 310 to the mounting post 320. The first coupler 342 may pass through an aperture 340 in mounting plate 310 and into a hole 324 in mounting post 320. The vertical hole 324 may be a blind and threaded hole as shown in FIG. 4. In other embodiments the hole 324 may not be blind (e.g., a through hole) and it may not be threaded. When hole 324 is threaded, coupler 342 may be a screw. For example, the coupler may be a socket head cap screw as depicted in the embodiment of FIG. 4. A washer 343 may be included between the coupler 342 and a shoulder within the aperture 340.

The second coupler 360 may couple the base 350 to the mounting post 320. The second coupler 360 may pass through an aperture 352 in base 350 and into a hole 326 in mounting post 320. The hole 326 may be a blind and threaded hole as shown in FIG. 4. In other embodiments the hole 326 may not be blind (e.g., a through hole) and it may not be threaded. In some embodiments, the holes 324, 326 may join to form a through hole. When hole 326 is threaded, coupler 360 may be a screw. For example, the coupler may be a socket head cap screw as depicted in the embodiment of FIG. 4.

The mounting plate 310 may also include apertures 332 configured to receive pins 330. Pins 330 are configured and/or adapted to receive a suture carrier and a cover, such as carrier 100 of FIG. 1 and cover 200 of FIG. 2. In some embodiments, such as the embodiment in FIGS. 3 and 4, the wrapping fixture 300 includes eight pins 330 in a substantially circular or regular octagonal arrangement such that the eight carrier holes 180 or eight cover apertures 210 may slip over the pins 330.

The mounting plate 310 may also include one or more bevels 312. When a suture carrier is placed on the fixture 300, the bevels 312 in the plate 310 create a space between the underside of a suture carrier and the mounting plate 310, which may allow a user to grip or otherwise handle the bottom of a suture carrier. This may facilitate handling a suture carrier, and in particular removing a suture carrier from the wrapping fixture 300.

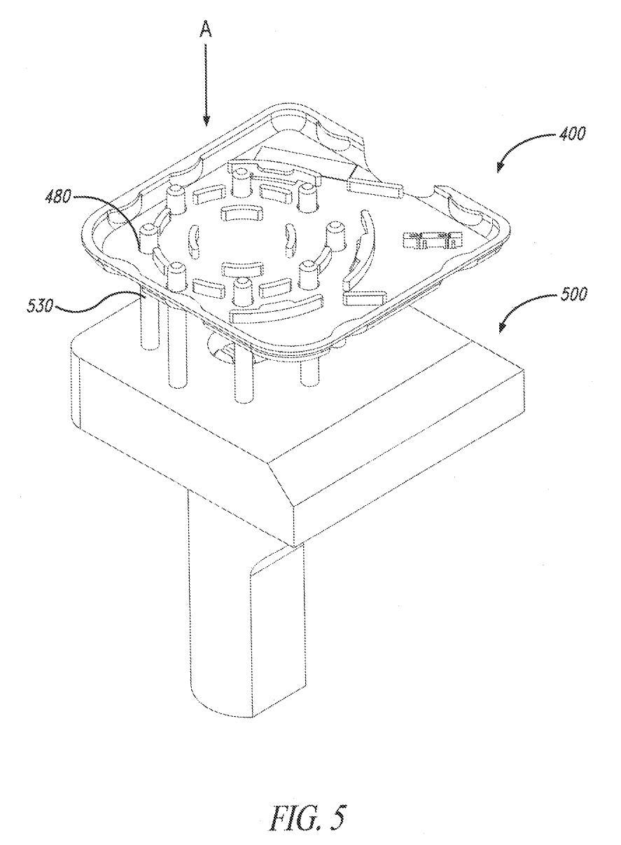

FIGS. 5 through 10 depict embodiments of various actions that may be involved in loading or packaging a suture in a suture carrier device.

FIG. 5 shows an embodiment of the actions for loading a suture carrier 400 onto a wrapping fixture 500. Wrapping fixture apertures 480 may be aligned with fixture pins 530, also called wrapping posts, and the carrier may be lowered onto the wrapping fixture in a downward direction, for example, direction A.

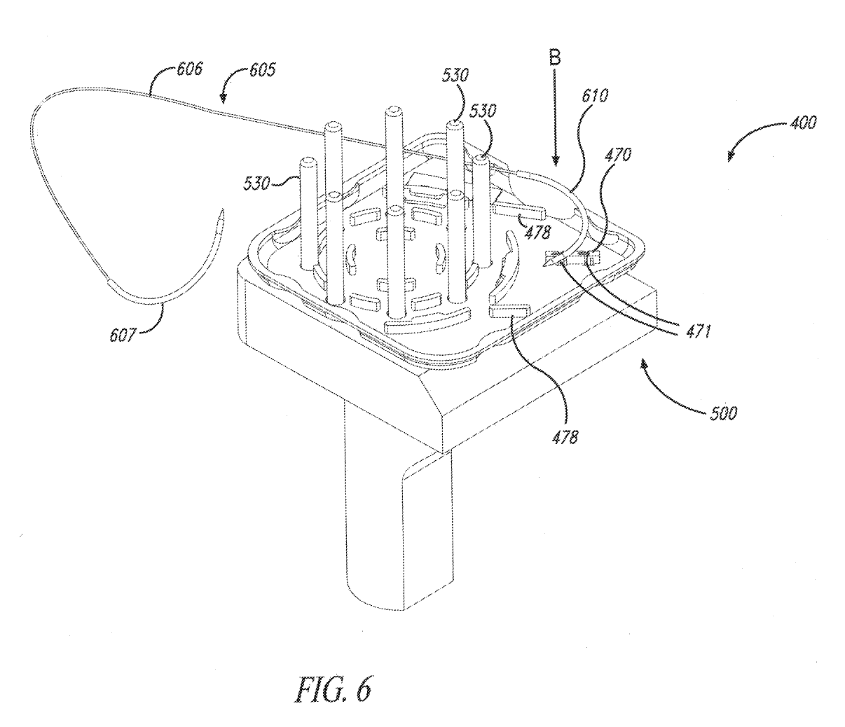

In FIG. 6, suture 605, may be loaded into the suture carrier 400. The suture 605 is a single suture with two needles 610, 607. The first suture needle 610may be secured in a needle retainer 470 by pressing the first needle 610into a respective needle retention slot 471. A force in direction B may be applied to the needle 610 to engage the needle within the needle retainer 470. The operator may also position the suture thread 606 to the outside of the fixture pins 530 projecting from the wrapping fixture 500.

FIG. 6 also depicts the needle retaining walls 478 guarding against the sharp ends of the suture needle 610.

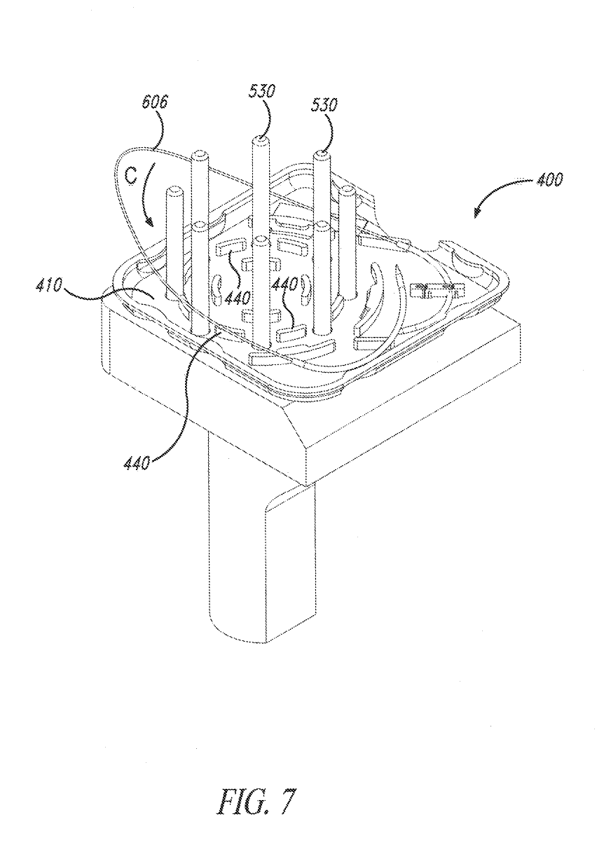

In FIG. 7, a user may wrap the suture thread 606 onto the fixture pins 530 and in a primary wrapping path 410 of the carrier. For example, a user wraps the suture thread 606 around the fixture pins 530 in a counter-clockwise direction C in a loop. In the embodiment shown in FIG. 7, the outside of the fixture pins 530 correspond to a primary wrapping path 410 of the suture carrier 400 such that by wrapping the suture thread 606 around the fixture pins 530, the user is wrapping the thread 606 along the primary wrapping path 410 and around the outside of the intermediate carrier walls 440.

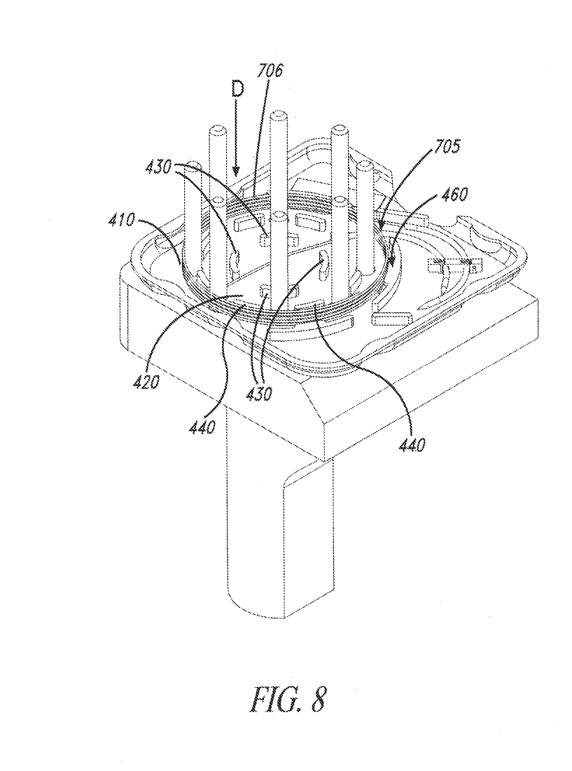

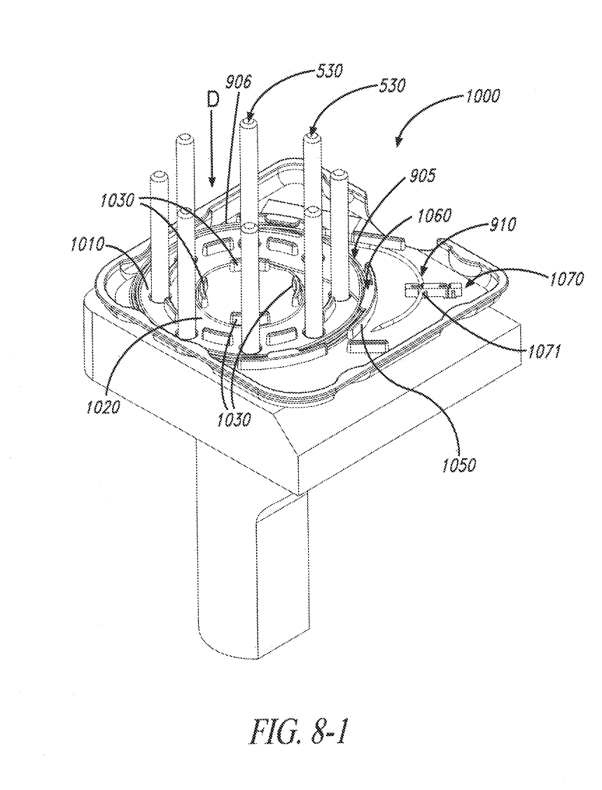

In FIG. 8, a user finishes wrapping the suture thread 606 in the primary wrapping path 410 and secures the second needle 607 into a respective needle retention slot 471 of the needle retainer 470.

In some embodiments, the end of the suture thread may be wrapped in other arrangements, for example, the suture thread may be wrapped around one or more inner carrier walls 430 and one or more intermediate carrier walls 440.

In some embodiments, a user may also position suture thread 706 under suture retaining canopies 460, or otherwise engage suture thread 706 in the retaining canopies 460, as shown in FIG. 8.

FIG. 8-1 shows an embodiment of a variable loop suture 905. In such an embodiment, a user first secures a needle 910 into a needle retention slot 1071 of a needle retainer 1070 on a suture carrier 1000. The user may then wrap suture thread 906 around fixture pins 530 and in the primary wrapping path 1010. While holding a portion of suture thread 906 in the primary wrapping path, a user may secure the variable loop end of the suture thread around the inner carrier walls 1030 and within a secondary wrapping path 1020.

In some embodiments, the suture may be a single suture with one needle or a suture without needles.

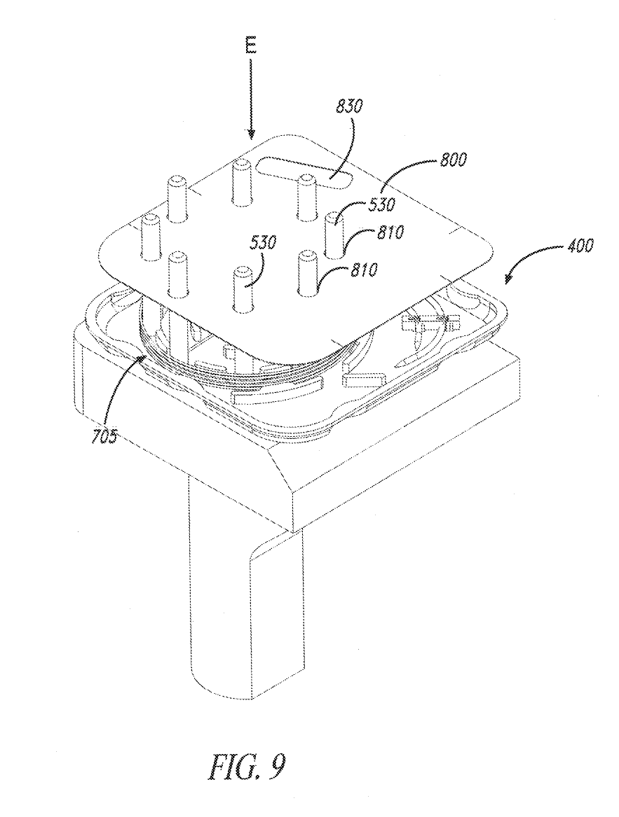

In FIG. 9 a user places a suture cover onto a wrapping fixture. While holding down the sutures 705, a user aligns a cover 800 with the carrier 400 by aligning the apertures 810 with the fixture pins 530 and a cutout 830, with a corresponding recess on the suture carrier, not shown. A user may slide the cover 800 along the fixture pins 530 in the direction E.

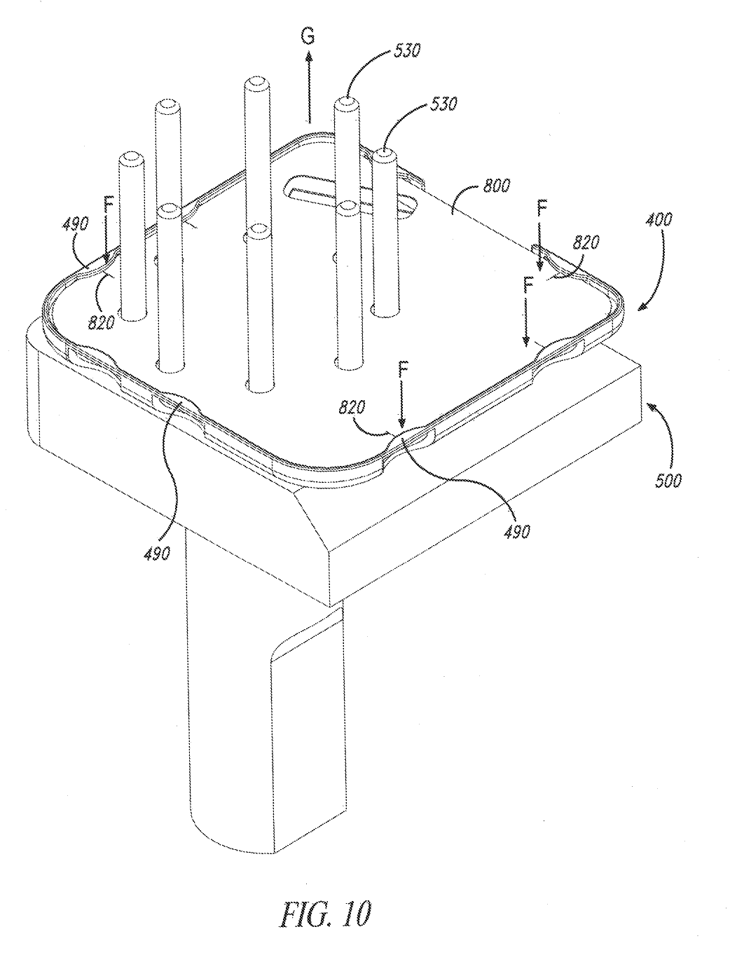

In FIG. 10, a user assembles a suture cover onto a suture carrier. After sliding the cover 800 down along the fixture pins 530, a user may secure the cover 800 onto the carrier 400 by pressing downward on the cover 800 in a direction F at the location of each suture cover tab 490 and a corresponding slit 820. The user may push the cover 800 causing it to pass past the cover tabs 490 to a potion underneath the cover tabs 490.

The user may also lift the assembled suture carrier 400 and cover 800 up off the wrapping fixture 500 in direction G. Once assembled, the suture carrier 400 may be processed or further packaged for use by medical professionals.

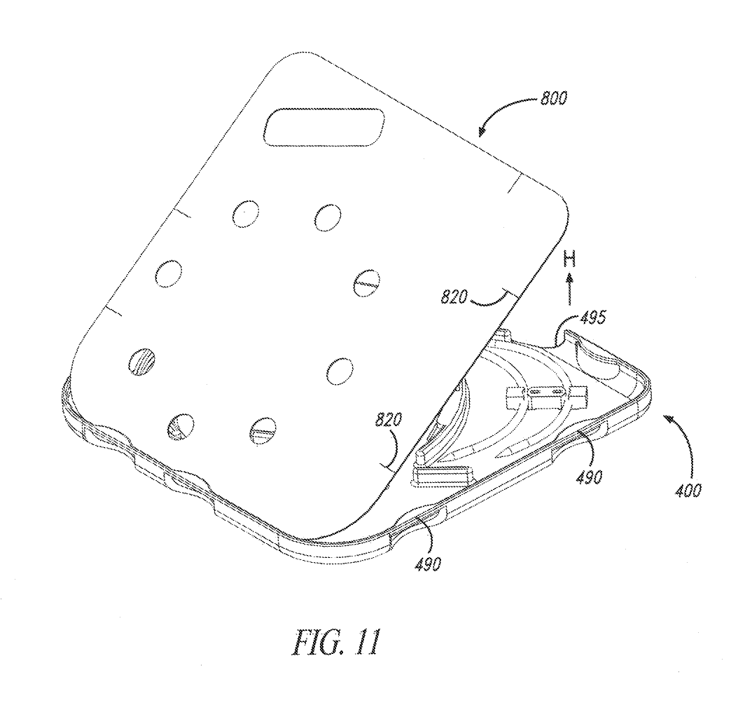

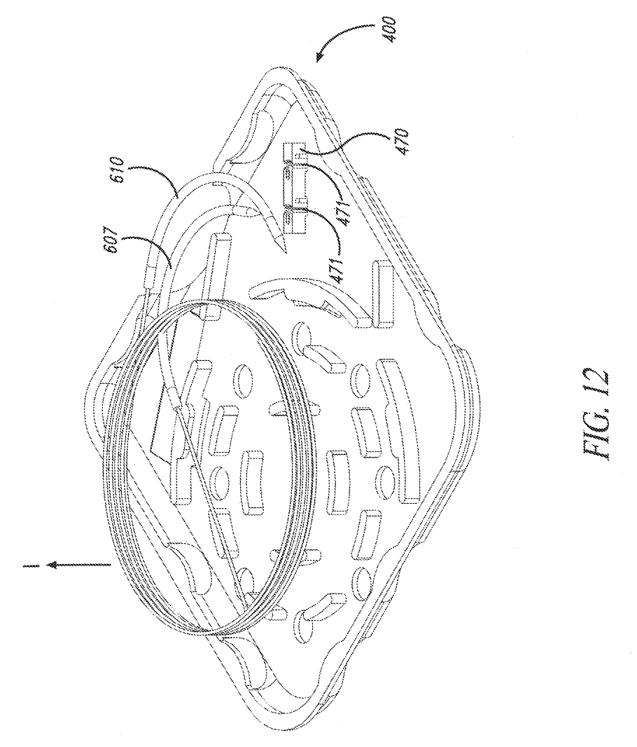

FIGS. 11 and 12 depict embodiments of various actions that may be involved in unloading or unpacking a suture in a suture carrier device. In FIG. 11, a user, such as a medical professional, may open the suture carrier 400 by gripping the cover 800 in a location corresponding to a cutout 495 in the suture carrier 400 and lifting up in a direction H. By lifting up, the cover is pulled past and freed from the cover tabs 490. The slits 820 can facilitate the removal of the cover 800 by providing weakened areas in the cover 800 that correspond to the cover tabs 490. The weakened cover 800 may require less force to remove from the suture carrier 400 than a cover without the through slits 820.

In FIG. 12, the user, such as a medical professional, may use forceps to remove the needles 710, 610 from the slots 471 of the needle retainer 470 and may lift the sutures from the carrier 400 in direction I.

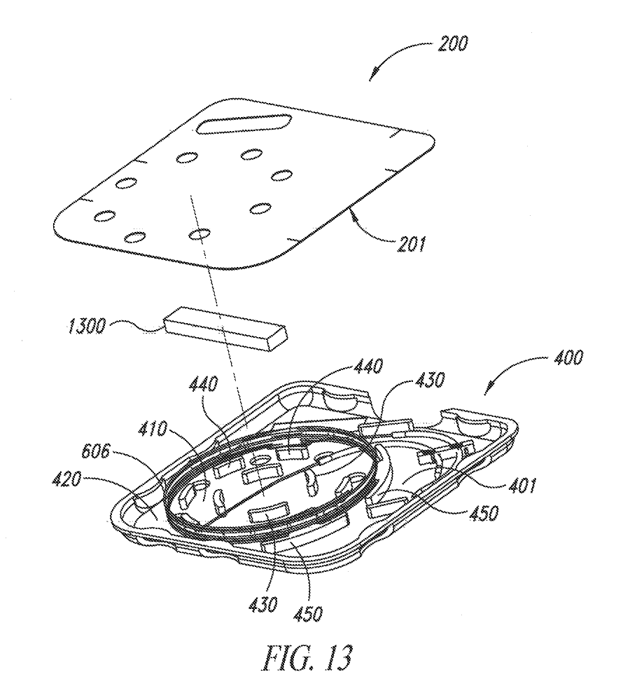

FIG. 13 depicts an embodiment of a suture carrier with a suture holding structure 1300. The suture holding structure 1300 may hold the suture thread 606 in place within the suture carrier 400 during transit. For example, in some embodiments, after wrapping the thread in the primary or secondary suture path, a user may place the suture holding structure 1300 on top of the suture threads 606 and then install the cover 200 onto the suture carrier 400 and over the suture holding structure 1300. The force between the cover 200 and a base 401 of the suture carrier 400 may hold the suture holding structure in place.

In some embodiments, the suture holding structure 1300 may be disposed on an underside 201 of the cover 200. The suture holding structure 1300 may be affixed or coupled to the underside 201 of the cover 200. In such an embodiment, the suture holding structure 1300 is placed over the suture threads 606 when the cover 200 is installed.

In some embodiments, the suture holding structure 1300 is configured with a length such that it extends over the secondary wrapping path 410 and may extend between two or more inner carrier walls 130. In some embodiments, the suture holding structure 1300 may extend over the primary wrapping path 420 and may also extend between intermediate carrier walls 430 or even outer carrier walls 450.

In some embodiments, the suture carrier 400 may include more than one suture holding structure 1300. For example, the suture carrier may include two or more suture holding structures 1300 positioned around one or more suture wrapping paths, but not passing through the center.

The suture holding structure may be made from open or closed cell foam or other material. The present disclosure provides the following exemplary embodiments:

1) An apparatus for holding a suture, the apparatus comprising: a carrier including a base, the base configured for being mounted on a fixture and including a primary wrapping path disposed thereon, the wrapping path adapted to receive at least a portion of a suture thread;

a plurality of suture retaining canopies located above an outer portion of the primary wrapping path and projecting outwardly from a portion of the base, the suture retaining canopies configured to facilitate retaining a suture thread within the primary wrapping path; and

a needle retainer disposed on the base, the needle retainer configured to retain a needle.

2) The apparatus of embodiment 1 wherein the base includes a secondary wrapping path for wrapping at least a portion of the suture thread.

3) The apparatus of embodiment 1, further comprising:

at least one outer carrier wall defining an outer boundary of the primary wrapping path.

4) The apparatus of embodiment 3 wherein the outer carrier wall is integrated with the base of the carrier and protrudes upward from the base.

5) The apparatus of embodiment 3 wherein at least one of the suture retaining canopies projects laterally from one of the at least one outer carrier walls.

6. The apparatus of embodiment 5 wherein at least one of the suture retaining canopies includes a wall projecting from one of the at least one outer carrier walls and over at least a portion of the primary wrapping path.

7) The apparatus of embodiment 2, further comprising:

at least one inner carrier wall defining an inner boundary of the secondary wrapping path, an intermediate carrier wall defining an outer boundary of the secondary wrapping path and an inner boundary of the primary wrapping path and an outer carrier wall defining an outer boundary of the primary wrapping path.

8) The apparatus of embodiment 1, further comprising:

at least two needle walls projecting from the base and located on opposite sides of the needle retainer.

9) The apparatus of embodiment 1, further comprising:

a suture carrier cover including a perimeter with a size and shape corresponding to a size and shape of a perimeter of the carrier and including at least one through slit extending from the perimeter of the cover.

10) The apparatus of embodiment 9, further comprising:

a plurality of cover tabs configured to secure a carrier cover by engaging the perimeter of the carrier cover.

11) The apparatus of embodiment 10 wherein the cover tabs project from a perimeter wall of the carrier, the perimeter wall projecting upwards from the base of the carrier.

12) The apparatus of embodiment 1 wherein the base includes a cut-out along an edge of the base, the cut-out configured to facilitate access to an underside of a cover.

13) The apparatus of embodiment 1 wherein the carrier is configured to retain a cover over the base.

14) The apparatus of embodiment 13, further comprising:

a suture retaining structure disposed on an underside of the cover.

15) A method of using a suture carrier, the method comprising:

loading a suture carrier onto a wrapping fixture by aligning one or more fixture apertures in the carrier with a corresponding one or more fixture pins on the fixture and lowering the suture carrier onto the wrapping fixture;

wrapping a first suture thread into a first loop and placing the first suture thread on a base of the suture carrier;

positioning at least a portion of the first loop of the first suture thread under a suture canopy;

installing a cover over the first suture and the suture carrier; and

engaging the cover with at least a portion of the suture carrier.

16) The method of embodiment 15 further comprising:

providing a needle retainer on the base of the suture carrier and outside an outer perimeter of a primary wrapping path; and

engaging a suture needle securely in the needle retainer.

17) The method of embodiment 15 wherein wrapping the suture thread into the first loop and placing the suture thread on the base of the suture carrier includes:

wrapping a first suture thread around the one or more fixture pins;

placing the first loop of first suture thread in a primary wrapping path;

wrapping the first suture thread to form a second loop; and

placing the second loop in a secondary wrapping path.

18) The method of embodiment 15 installing a cover over the suture and the suture carrier includes:

aligning one or more wrapping fixture apertures with a corresponding one or more alignment apertures provided through the cover and lowering the suture carrier onto the wrapping fixture.

19) The method of embodiment 15 wherein engaging the cover with at least a portion of the suture carrier includes:

engaging one or more portions of a perimeter of the cover with corresponding one or more cover tabs disposed on the base of the suture carrier.

20) The method of embodiment 19 further comprising:

grasping the cover in a location proximate to a cut-out provided in the suture carrier; and

lifting the cover such that the perimeter of the cover disengages from the cover tabs.

21) The method of embodiment 19 wherein engaging one or more portions of a perimeter of the cover with corresponding one or more cover tabs disposed on the base of the suture carrier includes:

applying a downward force in the cover at the one or more portions of the perimeter of the cover.

22) The method of embodiment 19, further comprising:

wrapping a second suture thread into the first loop with the first suture thread and placing the second suture thread on a base of the suture carrier with the first suture thread.

23) The method of embodiment 15 wherein the suture is a variable loop suture and further comprising:

securing a variable loop end of the suture around inner carrier walls.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An apparatus for holding a suture, the apparatus comprising:
a carrier including a base, the base configured for being mounted on a fixture and including a primary wrapping path disposed thereon, the wrapping path adapted to receive at least a portion of a suture thread;
a plurality of suture retaining canopies located above an outer portion of the primary wrapping path and projecting outwardly from a portion of the base, the suture retaining canopies configured to facilitate retaining a suture thread within the primary wrapping path; and
a needle retainer disposed on the base, the needle retainer configured to retain a needle.

2. The apparatus of claim 1 wherein the base includes a secondary wrapping path for wrapping at least a portion of the suture thread.

3. The apparatus of claim 1, further comprising:
at least one outer carrier wall defining an outer boundary of the primary wrapping path.

4. The apparatus of claim 3 wherein the outer carrier wall is integrated with the base of the carrier and protrudes upward from the base.

5. The apparatus of claim 3 wherein at least one of the suture retaining canopies projects laterally from one of the at least one outer carrier walls.

6. The apparatus of claim 5 wherein at least one of the suture retaining canopies includes a wall projecting from one of the at least one outer carrier walls and over at least a portion of the primary wrapping path.

7. The apparatus of claim 2, further comprising:
at least one inner carrier wall defining an inner boundary of the secondary wrapping path, an intermediate carrier wall defining an outer boundary of the secondary wrapping path and an inner boundary of the primary wrapping path and an outer carrier wall defining an outer boundary of the primary wrapping path.

8. The apparatus of claim 1, further comprising:
at least two needle walls projecting from the base and located on opposite sides of the needle retainer.

9. The apparatus of claim 1, further comprising:
a suture carrier cover including a perimeter with a size and shape corresponding to a size and shape of a perimeter of the carrier and including at least one through slit extending from the perimeter of the cover.

10. The apparatus of claim 9, further comprising:
a plurality of cover tabs configured to secure a carrier cover by engaging the perimeter of the carrier cover.

11. The apparatus of claim 10 wherein the cover tabs project from a perimeter wall of the carrier, the perimeter wall projecting upwards from the base of the carrier.

12. The apparatus of claim 1 wherein the base includes a cut-out along an edge of the base, the cut-out configured to facilitate access to an underside of a cover.

13. The apparatus of claim 1 wherein the carrier is configured to retain a cover over the base.

14. The apparatus of claim 13, further comprising:
a suture retaining structure disposed on an underside of the cover.

15. A method of using a suture carrier, the method comprising:
loading a suture carrier onto a wrapping fixture by aligning one or more fixture apertures in the carrier with a corresponding one or more fixture pins on the fixture and lowering the suture carrier onto the wrapping fixture;
wrapping a first suture thread into a first loop and placing the first suture thread on a base of the suture carrier;
positioning at least a portion of the first loop of the first suture thread under a suture canopy;
installing a cover over the first suture and the suture carrier; and
engaging the cover with at least a portion of the suture carrier.

16. The method of claim 15 further comprising:
providing a needle retainer on the base of the suture carrier and outside an outer perimeter of a primary wrapping path; and
engaging a suture needle securely in the needle retainer.

17. The method of claim 15 wherein wrapping the suture thread into the first loop and placing the suture thread on the base of the suture carrier includes:
wrapping a first suture thread around the one or more fixture pins;
placing the first loop of first suture thread in a primary wrapping path;
wrapping the first suture thread to form a second loop; and
placing the second loop in a secondary wrapping path.

18. The method of claim 15 installing a cover over the suture and the suture carrier includes:
aligning one or more wrapping fixture apertures with a corresponding one or more alignment apertures provided through the cover and lowering the suture carrier onto the wrapping fixture.

19. The method of claim 15 wherein engaging the cover with at least a portion of the suture carrier includes:
engaging one or more portions of a perimeter of the cover with corresponding one or more cover tabs disposed on the base of the suture carrier.

20. The method of claim 19 further comprising:
grasping the cover in a location proximate to a cut-out provided in the suture carrier; and
lifting the cover such that the perimeter of the cover disengages from the cover tabs.

21.-23. (canceled)
